(19) United States
(12) Patent Application Publication
(10) Pub. No.: US 2026/0083537 A1
(43) Pub. Date: Mar. 26, 2026

(54) ORAL RETRACTOR WITH INTEGRATED SUCTION

(71) Applicant: Prashanthi Baddam DMD PC, Mesa, AZ (US)

(72) Inventor: Prashanthi Baddam, Mesa, AZ (US)

(73) Assignee: Prashanthi Baddam DMD PC, Mesa, AZ (US)

(21) Appl. No.: 18/893,759

(22) Filed: Sep. 23, 2024

(51) Int. Cl. A61B 1/24; A61C 19/00

(52) U.S. Cl. CPC A61C 17/10; A61B 13/00

(57) ABSTRACT

An oral retractor with integrated suction. The oral retractor has a hollow elongated body, an angled junction extending from the elongated body, and a solid blunt working end extending from the angled junction. The oral retractor has a series of small circular apertures formed on each of two outer side surfaces of the angled junction. Alternatively, the oral retractor has one elongated aperture formed on each of two outer side surfaces of the angled junction. A suction hose is coupled to a proximal end of the oral retractor so that water, debris, and saliva may be evacuated from the patient's oral cavity. The water, debris, and saliva travel through the aperture(s) formed on the two outer side surfaces of the angled junction, through a channel contained within the angled junction and elongated body, and will exit through an opening in the rear connection end of the oral retractor.

### FIELD OF THE INVENTION

The present invention relates generally to the field of dentistry. More specifically, the present invention is an oral retractor with integrated suction capability.

### BACKGROUND OF THE INVENTION

During routine dental procedures or oral surgery, oral retractors are used to move the patient's cheeks, lips, tongue, and any other oral tissues out of the way. Usually, the dentist, oral surgeon, or dental hygienist will use an oral retractor as well as a separate suction device to remove any fluid or debris from the mouth during the procedure. The suction device is sometimes bent like a hook and left hanging hands-free out of the patient's mouth. This hands-free positioning is not effective because it is not positioned properly within the oral cavity. Furthermore, it can easily be knocked out of place and fall onto the floor, thus requiring replacement or sterilization prior to being reinserted into the patient's mouth. Alternatively, an additional person may be needed during the procedure so that there are enough hands to hold all of the dental tools. This arrangement is also inefficient because the oral cavity is already a small work space and any additional hands or tools crowds the area.

Some currently used oral retractors have holes in them and these holes are intended to be used to remove debris from the oral cavity during the dental procedure. However, the holes of those oral retractors are positioned on the working end of those oral retractors. The placement of the holes on the working end of those oral retractors is ineffective because these holes are often blocked by surrounding mouth tissues.

The present disclosure provides for an oral retractor with integrated suction capability. Rather than having to use two different tools for retraction and suction, the present invention provides a more streamlined and efficient device. The present disclosure also provides for an oral retractor with one or more apertures positioned above or proximal to its working end and positioned on the side surfaces of the device; this particular positioning of the aperture(s) helps prevent the aperture(s) from being blocked by surrounding mouth tissues. Other benefits and advantages will become clear from the disclosure provided herein and those advantages provided are for illustration. The statements in this section merely provide the background related to the present disclosure and do not constitute prior art.

### SUMMARY

This summary is provided to introduce a selection of concepts in a simplified form that are further described below in the DESCRIPTION OF THE DISCLOSURE. This summary is not intended to identify key features of the claimed subject matter, nor is it intended to be used as an aid in determining the scope of the claimed subject matter.

In accordance with one or more embodiments of the present invention, an oral retractor is disclosed. The oral retractor comprises: a hollow elongated body; an angled junction extending from a distal end of the elongated body; a solid blunt working end extending from a distal point of the angled junction; and at least two apertures formed on the angled junction.

In accordance with one or more embodiments of the present invention, an oral retractor is disclosed. The oral retractor oral retractor comprises: a hollow elongated body; an angled junction extending from a distal end of the elongated body; a solid blunt working end extending from a distal point of the angled junction; and at least one aperture formed on each of two outer side surfaces of the angled junction.

In accordance with one or more embodiments of the present invention, an oral retractor is disclosed. The oral retractor comprises: a hollow elongated body; an angled junction extending from a distal end of the elongated body; a solid blunt working end extending from a distal point of the angled junction, the blunt working end being curved and comprising: a concave center portion; and two wings extending outwardly in opposing directions from the center portion, wherein an outermost point of each wing is positioned at about a 20° angle with respect to the center portion of the blunt working end; a channel beginning at a rear connection end of the elongated body, extending through a length of the elongated body, and terminating at the distal point of the angled junction; and at least one aperture formed on each of two outer side surfaces of the angled junction, the at least one aperture being contiguous with and perpendicular to the channel extending through the length of the elongated body.

### BRIEF DESCRIPTION OF THE DRAWINGS

The novel features believed to be characteristic of the disclosure are set forth in the appended claims. In the descriptions that follow, like parts are marked throughout the specification and drawings with the same numerals, respectively. The drawing FIGURES are not necessarily drawn to scale and certain FIGURES may be shown in exaggerated or generalized form in the interest of clarity and conciseness. The disclosure itself, however, as well as a preferred mode of use, further objectives and advantages thereof, will be best understood by reference to the following detailed description of illustrative embodiments when read in conjunction with the accompanying drawings, wherein:

FIG. 1 is a perspective view of an oral retractor with integrated suction in accordance with one aspect of the present disclosure, shown in use;

FIG. 2 is a rear elevated perspective view of the oral retractor of FIG. 1;

FIG. 3 is top view of the oral retractor of FIG. 1;

FIG. 4 is a bottom view of the oral retractor of FIG. 1;

FIG. 5 is a right side view of the oral retractor of FIG. 1;

FIG. 6 is a left side view of the oral retractor of FIG. 1;

FIG. 7 is a right side cross-sectional view taken along line 7-7 of FIG. 5;

FIG. 8 is a rear view of the oral retractor of FIG. 1;

FIG. 9 is a front view of the oral retractor of FIG. 1;

FIG. 10 is a rear elevated perspective view of an oral retractor with integrated suction in accordance with another aspect of the present disclosure;

FIG. 11 is top view of the oral retractor of FIG. 10;

FIG. 12 is a bottom view of the oral retractor of FIG. 10;

FIG. 13 is a right side view of the oral retractor of FIG. 10;

FIG. 14 is a left side view of the oral retractor of FIG. 10;

FIG. 15 is a right side cross-sectional view taken along line 15-15 of FIG. 13;

FIG. 16 is a rear view of the oral retractor of FIG. 10; and

FIG. 17 is a front view of the oral retractor of FIG. 10.

### DETAILED DESCRIPTION OF THE INVENTION

The description set forth below in connection with the appended drawings is intended as a description of exemplary embodiments of the disclosure and is not intended to represent the only forms in which the present disclosure may be constructed and/or utilized. The description sets forth the functions and the sequence of blocks for constructing and operating the disclosure in connection with the illustrated embodiments. It is to be understood, however, that the same or equivalent functions and sequences may be accomplished by different embodiments that are also intended to be encompassed within the spirit and scope of this disclosure.

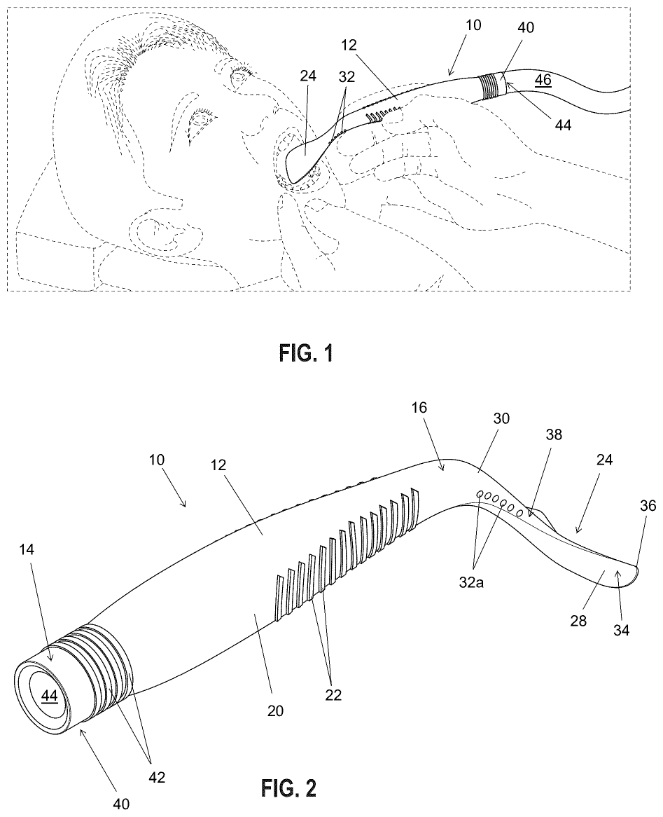

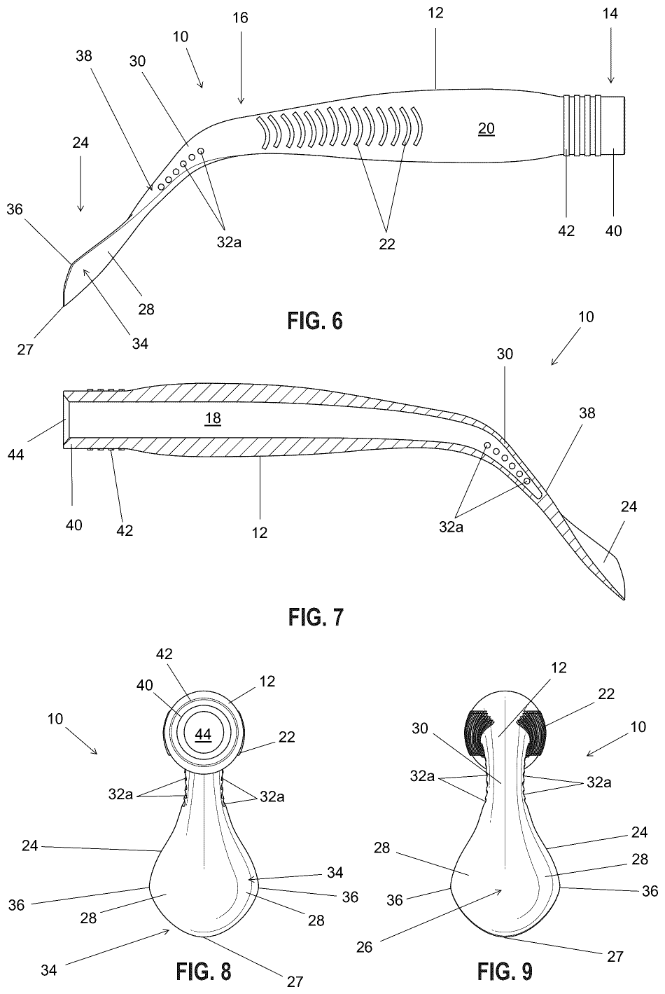

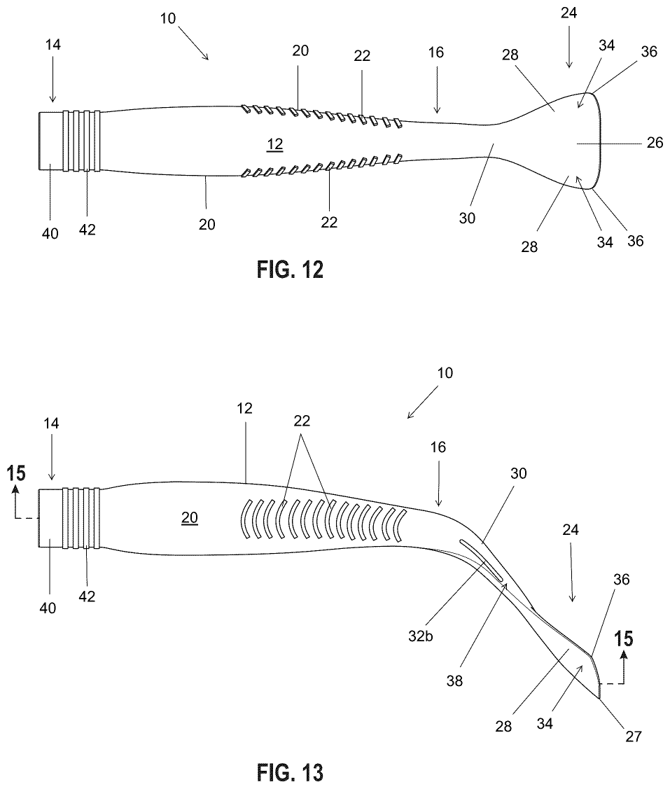

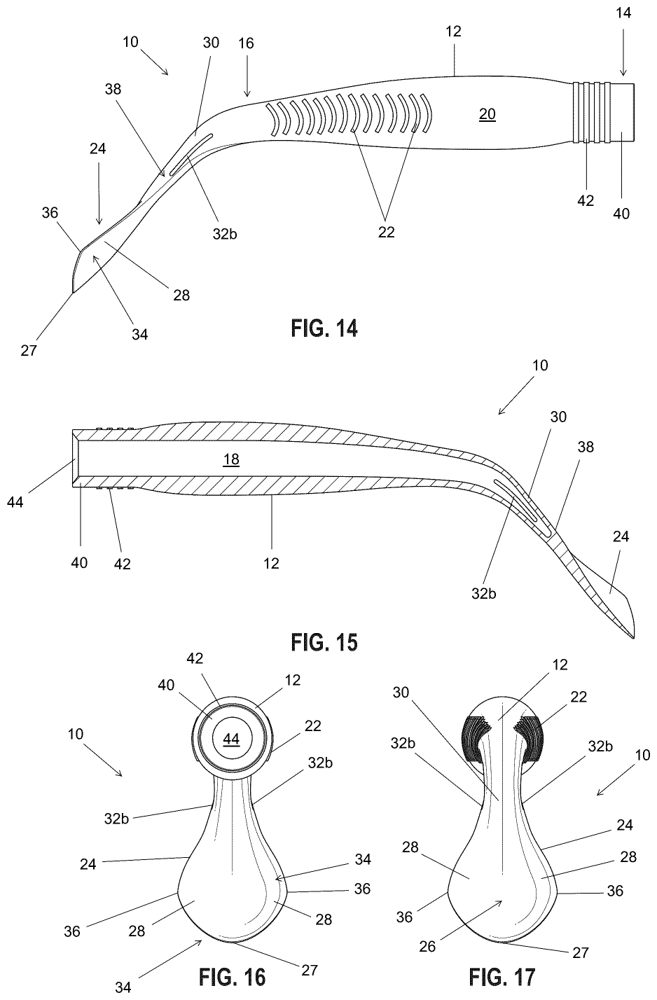

FIGS. 1-17 together disclose an oral retractor with integrated suction capability, hereinafter referred to as oral retractor 10. In its simplest form, the oral retractor 10 has a hollow elongated body 12 with a blunt working end 24 formed at its distal end 16 and at least one aperture 32 formed on the angled junction 30 where the elongated body 12 and the blunt working end 24 meet.

FIG. 1 shows the oral retractor 10 in use with a dental patient. The blunt working end 24 of the oral retractor 10 is shown being inserted into the patient's oral cavity. The oral retractor 10 has an opening 44 formed in its rear connection end 40. The opening 44 may be circular or any other suitable shape as long as it is configured to be coupled to a suction hose 46. One end of a suction hose 46 is shown inserted into the opening 44 formed in the rear connection end 40 of the oral retractor 10, creating a friction fit. It should be clearly understood that other forms for connections may be used between the suction hose 46 and the opening 44 formed in the rear connection end 40 of the oral retractor 10; e.g. snap fit ends, mated threaded ends, etc.

Referring to FIGS. 2-7, the oral retractor 10 has an elongated body 12, a blunt working end 24, and an angled junction 30 between the elongated body 12 and the blunt working end 24. At the angled junction 30, the blunt working end 24 may form an angle of about 48.5° with the elongated body 12. While the 48.5° angle is preferred, it should be clearly understood that substantial benefit may still be derived if the angle formed at the angled junction 30 is between 0°-90°.

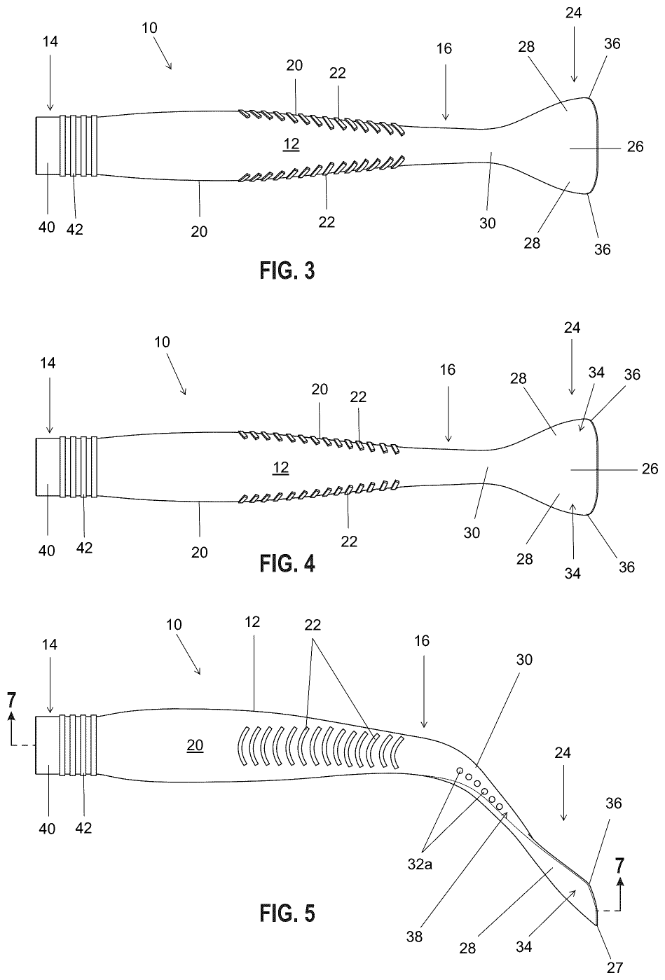

The width/diameter of the elongated body 12 may vary at different locations along its length. Referring to FIGS. 3-4, the outer side surfaces 20 of the elongated body 12 may be parallel to each other near its proximal end 14. Then the elongated body 12 may gradually increase in diameter as it approaches a mid-length point on the elongated body 12; this mid-length point may be where the plurality of ridges 22 (discussed below) begin. Beyond the mid-length point on the elongated body 12, the diameter of the elongated body 12 may gradually taper and become narrower near its distal end 16 as it approaches the angled junction 30. The narrowest part of the oral retractor 10 may be the angled junction 30.

As shown in FIG. 7, the elongated body 12 of the oral retractor 10 is hollow, while the blunt working end 24 of the oral retractor 10 is entirely solid with no apertures or channels therein. The elongated body 12 has a channel 18 formed through its center. The channel 18 is contiguous and aligned with the opening 44 formed in the rear connection end 40 of the oral retractor 10, extends through the length of the elongated body 12, and terminates at a distal point 38 of the angled junction 30, where the angled junction 30 ends and the blunt working end 24 begins. The entire channel 18 is contained within the elongated body 12 and the angled junction 30 of the oral retractor 10.

FIGS. 2 and 5-7 show that the oral retractor 10 has a plurality of apertures 32a (referred to generically as aperture 32) formed in a single line along the length of the angled junction 30. The apertures 32a are formed on the outer side surfaces 20 of the oral retractor 10 and located at the angled junction 30. The positioning of the apertures 32 on the outer side surfaces 20 of the oral retractor 10 helps to ensure that the apertures 32 are not blocked by the surrounding tissues of the patient's oral cavity. If the apertures 32 are blocked by the surrounding tissues, then the liquid, saliva, and debris cannot be effectively removed from the oral cavity during the dental procedure. While it is shown that six apertures 32a are formed along the length of the angled junction 30, it should be clearly understood that substantial benefit may be derived from the use of any suitable number of apertures 32a. In this embodiment, the apertures 32a are circular apertures that are contiguous with and positioned perpendicular to the channel 18 that extends through the length of the elongated body 12. The circular apertures 32a may each have a diameter of about 1.5 mm, but it should be clearly understood that substantial benefit may still be derived from the circular apertures 32a having a diameter that is larger or smaller than 1.5 mm as long as the circular apertures 32a provide effective evacuation of water, saliva, and debris from the surgical area. It should also be clearly understood that the circular apertures 32a may have an alternate shape; e.g. oval, square, triangular, etc. While the drawings shown that the circular apertures 32a may have the same diameter, it should be clearly understood that substantial benefit may also be derived from the circular apertures 32a having diameters of varying sizes.

In FIGS. 1-6, the oral retractor 10 is shown as having a plurality of ridges 22 formed on the outer side surfaces 20 of the oral retractor 10. These ridges 22 are positioned about halfway along the length of the elongated body 12 and continue along the length of the elongated body 12 toward the distal end 16 of the elongated body 12. These ridges 22 may help to provide a non-slip area for the dentist, surgeon, or hygienist to grip while using the oral retractor 10. Although the ridges 22 are shown as having a curved shape, it should be clearly understood that the ridges 22 may have any suitable shape that will provide a non-slip surface for gripping. And although the ridges 22 are shown as only being formed on the outer side surfaces 20 of the elongated body 12, it should be clearly understood that substantial benefit may be derived from the ridges 22 being formed around the entire circumference of the elongated body 12.

The oral retractor 10 may have a plurality of annular ridges 42 formed on the outer surface of the elongated body 12. As shown in FIGS. 2-8, the annular ridges 42 may be positioned near the proximal end 14 of the elongated body 12 and shaped to surround the opening 44 in the rear connection end 40. These annular ridges 42. These annular ridges 42 help to provide a non-slip area for the dentist, surgeon, or hygienist to grip while connecting the suction hose 46 to the opening 44 or disconnecting the suction hose 46 from the opening 44. Although the oral retractor 10 is shown as having a plurality of annular ridges 42, it should be clearly understood that substantial benefit may be derived from the oral retractor 10 having any suitable number of annular ridges 42.

The blunt working end 24 of the oral retractor 10 is the end that is inserted into the oral cavity of the patient. Referring to FIGS. 2-9, the blunt working end 24 has a center portion 26 with two wings 28 that extend outwardly and in opposing directions from the center portion 26. The center portion 26 may be flat or concave in shape. Each of the wings 28 gradually curves upwardly as it extends outwardly from the center portion 26 of the blunt working end 24; thus, the outermost point 36 of each wing 28 is positioned at an angle with respect to the center portion 26 of the blunt working end 24. Particularly, the outermost point 36 of each wing 28 is positioned at about a 20° angle with respect to the center portion 26 of the blunt working end 24. While 20° may be described, it should be clearly understood that substantial benefit may be derived from each wing 28 being positioned at an angle greater than or less than 20° with respect to the center portion 26, as long as the blunt working end 24 has an overall curved shape. The angle may vary, for example, if the oral retractor 10 is designed for use with a child patient versus an adult patient. This curvature of the blunt working end 24 allows the blunt working end 24 to fit more comfortably inside the patient's oral cavity compared to the currently used oral retractors. The oral retractors that are currently being used have a flat shaped working end, which causes the working end of those oral retractors to painfully dig into the soft palate, hard palate, and other tissues in the roof of the patient's mouth. The curvature of the blunt working end 24 of the oral retractor 10 of the present invention mimics the curvature of the roof of the patient's mouth, allowing a portion of a bottom surface 34 of the wings 28 to sit flush with the roof of the patient's mouth. This curvature of the blunt working end 24 therefore prevents the wings 28 from digging into the roof of the patient's mouth.

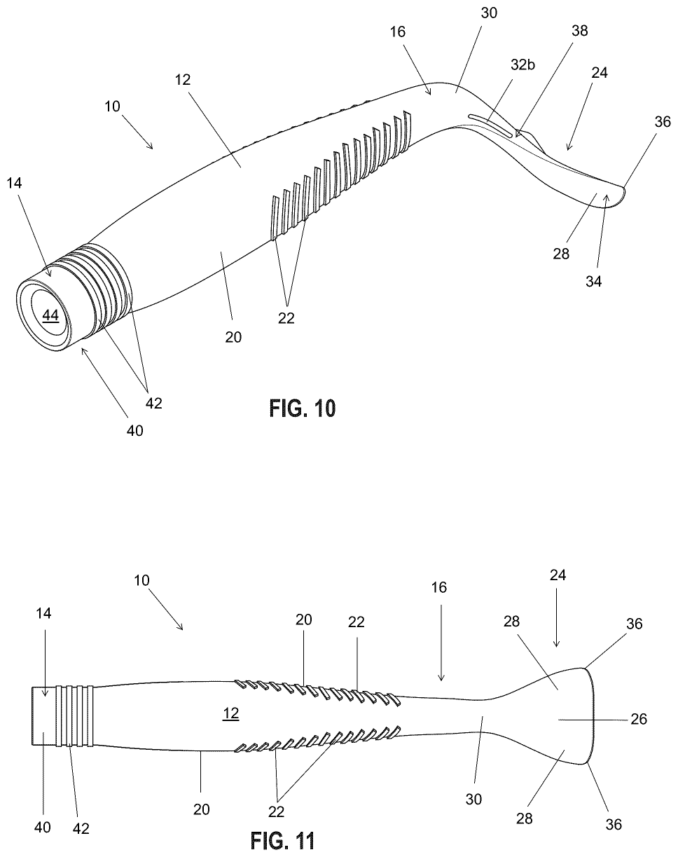

FIGS. 10-17 show another embodiment of the oral retractor 10 of the present invention. This embodiment is identical in structure to the oral retractor 10 shown in FIGS. 1-9, except for the form of the aperture 32. As shown in FIGS. 10 and 13-15, this embodiment of the oral retractor 10 has a single elongated aperture 32b (referred to generically as aperture 32) formed along the length of the angled junction 30 rather than a plurality of circular apertures 32a. The elongated aperture 32b is contiguous with and positioned perpendicular to the channel 18 that extends through the length of the elongated body 12. The elongated aperture 32b may have a length of about 15.787 mm and have a width of about 0.902 mm, but it should be clearly understood that substantial benefit may still be derived from the elongated aperture 32b having a length that is longer or shorter than 15.787 mm, as long as the elongated aperture 32b provides effective evacuation of water, saliva, and debris from the surgical area. It should also be clearly understood that substantial benefit may still be derived from the elongated aperture 32b having a width that is wider or narrower than 0.902 mm, as long as the elongated aperture 32b provides effective evacuation of water, saliva, and debris from the surgical area.

One example of the oral retractor 10 may have the following dimensions. These dimensions are for illustrative purposes only and the invention should not be limited to this particular example. The total length of the oral retractor 10 from the rear connection end 40 to the most distal point 27 of the blunt working end 24 may be 150.728 mm, with the blunt working end 24 itself being 35.534 mm long and with the elongated body 12 and angled junction 30 together making up the remaining length of 115.194 mm. The height/diameter of the elongated body 12 near its cylindrical proximal end 14 may be 15.566 mm. Then the height (from top surface to bottom surface) of the elongated body 12 and the width (from one side surface to the other side surface) of the elongated body 12 may gradually increase to a height of 19.465 mm and a width of 19.607 mm as it approaches a mid-length point on the elongated body 12; this mid-length point may be where the plurality of ridges 22 begin to appear. Beyond the mid-length point on the elongated body 12, the height of the elongated body 12 and the width of the elongated body 12 may gradually taper and decrease to a height of 3.736 mm and a width of 8.683 mm near the distal end 38 of the angled junction 30. From the angled junction 30, the width of the blunt working end 24 then gradually increases to 26.416 mm (the distance between the two outermost points 36 of the wings 28). The overall height of the oral retractor 10 from the rear connection end 40 to the most distal point of the blunt working end 24 may be 58.741 mm. The diameter of the opening 44 in the rear connection end 40 may be 10 mm. In the embodiment having a plurality of circular apertures 32a formed along the length of the angled junction 30, each circular aperture 32a may have a diameter of about 1.5 mm. In the embodiment having one elongated aperture 32b formed along the length of the angled junction 30, the elongated aperture 32b may have a length of about 15.787 mm and have a width of about 0.902 mm. The oral retractor 10 may be made of stainless steel, hard plastic, or any other suitable material as long as the oral retractor 10 may be autoclaved for easy and convenient sterilization.

When using the oral retractor 10 of the present invention, the surgeon, dentist or hygienist will connect a suction hose 46 to the rear connection end 40 of the oral retractor 10. During the procedure, water, saliva, and small debris will be sucked from the patient's oral cavity and will pass through the aperture(s) 32 of the angled junction 30 and through the channel 18 of the elongated body and will exit the oral retractor 10 through the opening 44 at the rear connection end 40. The apertures(s) 32 formed on the outer side surfaces 20 of the oral retractor 10 and positioned on the angled junction 30 (above/proximal to the blunt working end 24) will not be blocked or impeded by surrounding tissues in the patient's oral cavity, thus allowing for effective evacuation of water, saliva, and small debris from the oral cavity during the dental procedure.

The foregoing description is provided to enable any person skilled in the relevant art to practice the various embodiments described herein. Various modifications to these embodiments will be readily apparent to those skilled in the relevant art and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the relevant art are expressly incorporated herein by reference and intended to be encompassed by the claims. Moreover, nothing disclosed herein is intended to be dedicated to the public regardless of whether such disclosure is explicitly recited in the claims.

What is claimed is:

1. An oral retractor comprising:
a hollow elongated body;
an angled junction extending from a distal end of the elongated body;
a solid blunt working end extending from a distal point of the angled junction; and
at least two apertures formed on the angled junction.

2. The oral retractor of claim 1, wherein the elongated body comprises:
an opening formed within a rear connection end of the elongated body; and
a channel extending through a length of the elongated body, wherein the channel is contiguous with the opening formed within the rear connection end of the elongated body.

3. The oral retractor of claim 2, wherein the channel extending through the length of the elongated body terminates at the distal point of the angled junction.

4. The oral retractor of claim 1, wherein the blunt working end and the elongated body form an angle of about 48° at the angled junction.

5. The oral retractor of claim 1, wherein the blunt working end is curved.

6. The oral retractor of claim 5, wherein the blunt working end comprises:
a center portion; and
two wings extending outwardly in opposing directions from the center portion.

7. The oral retractor of claim 6, wherein the center portion is concave.

8. The oral retractor of claim 6, wherein an outermost point of each wing is positioned at about a 20° angle with respect to the center portion of the blunt working end.

9. The oral retractor of claim 1, wherein the angled junction has two side surfaces and the at least two apertures formed on the angled junction comprises a plurality of apertures formed on each outer side surface, the apertures being contiguous with and perpendicular to the channel extending through the length of the elongated body.

10. The oral retractor of claim 9, wherein the plurality of apertures on each outer side surface are positioned in a single line along a length of the angled junction.

11. The oral retractor of claim 9, wherein the plurality of apertures are circular shaped.

12. The oral retractor of claim 1, wherein the angled junction has two side surfaces and the at least two apertures formed on the angled junction comprises one elongated aperture formed on each outer side surface, the elongated aperture being contiguous with and perpendicular to the channel extending through the length of the elongated body.

13. The oral retractor of claim 1, wherein the at least two apertures formed on the angled junction are positioned proximal to the blunt working end.

14. An oral retractor comprising:
a hollow elongated body;
an angled junction extending from a distal end of the elongated body;
a solid blunt working end extending from a distal point of the angled junction; and
at least one aperture formed on each of two outer side surfaces of the angled junction.

15. The oral retractor of claim 14, wherein the elongated body comprises:
an opening formed within a rear connection end of the elongated body; and
a channel being contiguous with the opening formed within the rear connection end of the elongated body, extending through a length of the elongated body, and terminating at the distal point of the angled junction,
wherein the channel is entirely contained within the elongated body and the angled junction.

16. The oral retractor of claim 14, wherein the blunt working end and the elongated body form an angle of about 48° at the angled junction.

17. The oral retractor of claim 14, wherein the blunt working end is curved and comprises:
a concave center portion; and
two wings extending outwardly in opposing directions from the center portion,
wherein an outermost point of each wing is positioned at about a 20° angle with respect to the center portion of the blunt working end.

18. The oral retractor of claim 14, wherein the at least one aperture formed on each of two outer side surfaces comprises a plurality of circular apertures, the circular apertures being positioned in a single line along a length of the angled junction, being positioned proximal to the blunt working end, and being contiguous with and perpendicular to the channel extending through the length of the elongated body.

19. The oral retractor of claim 14, wherein the at least one aperture formed on each of two outer side surfaces comprises one elongated aperture, the elongated aperture being positioned proximal to the blunt working end and being contiguous with and perpendicular to the channel extending through the length of the elongated body.

20. An oral retractor comprising:
a hollow elongated body;
an angled junction extending from a distal end of the elongated body;
a solid blunt working end extending from a distal point of the angled junction, the blunt working end being curved and comprising:
a concave center portion; and
two wings extending outwardly in opposing directions from the center portion, wherein an outermost point of each wing is positioned at about a 20° angle with respect to the center portion of the blunt working end;
a channel beginning at a rear connection end of the elongated body, extending through a length of the elongated body, and terminating at the distal point of the angled junction; and at least one aperture formed on each of two outer side surfaces of the angled junction, the at least one aperture being contiguous with and perpendicular to the channel extending through the length of the elongated body.
